(19) United States
(12) Patent Application Publication
(10) Pub. No.: US 2005/0244524 A1
(43) Pub. Date: Nov. 3, 2005

(54) Blood sugar regulating product from soybean seeds

(76) Inventor: Manju Pathak, Noida (IN)

Correspondence Address: Dr. Manju Pathak;B-506, PMO Housing Society, C-58/20, Sector-62, Noida, Uttar Pradesh, 201301, IN

(21) Appl. No.: 11/102,935

(22) Filed: Apr. 7, 2005

Related U.S. Application Data
(63) Continuation of application No. PCT/IN04/00122, filed on Apr. 30, 2004.

(51) Int. Cl. A61K 35/78

(52) U.S. Cl. 424/757

(57) ABSTRACT

This invention is about the production/enhancement of the blood sugar regulating property in soybean seeds due to embryo development (soaking and germination) of the seeds and extraction and isolation of certain important phytochemicals to achieve the effective blood sugar regulating products. It is about the importance of the PI3K synthesized in the soybean root and root nodule. Soaking and germination of Soybean seeds produce high blood sugar regulating product, in this Invention.

### CROSS REFERENCE TO RELATED APPLICATIONS

My Indian patent no. 904/DEL/2002 and 663/DEL/2003, International Application number: PCT/IN2004/000122, International filing date: 30 Apr. 2004, International patent classification: A61K 35/78 and International publication number: WO 2004/096250 A1.

### STATEMENT REGARDING FEDERALLY SPONSORED RESEARCH ORDEVELOPMENT

“Not applicable”.

### REFERENCE TO SEQUENCE LISTING, A TABLE OR A COMPUTER PROGRAM LISTING COMPACT DISC APPENDIX ATTACHED HEREWITH ON PAPER

References and tables of observations are attached in typed form

### BACKGROUND OF THE INVENTION

Diabetes is a disease in which the sugar concentration of the blood increases. This increase in the sugar level of the blood causes many other physiological problems. Some of the physiological problems become fatal while others reduce the life span of the individual by 10 to 15 years and in certain cases by even more than this. The exact reason for diabetes is not yet clearly known. But its apparent cause and its control give us indication for some of the probable factors like increase in stress, change in lifestyle, malnourished diet and increase of fatty acids in the body, which create resistance in Insulin action. Diabetes is broadly classified in two types, type-1 and type-2. Type-1 diabetes is an auto-immune disease which is caused by total destruction of insulin producing pancreatic cells while type-2 is a late onset disease caused by the resistance faced by insulin. Till today diabetes is mainly controlled by allopathic medicines, which comprise oral drugs and insulin injections. But unfortunately these medicines are not able to control diabetes perfectly, rather they increase the severity of the disease and many times the side effects of these drugs are fatal. I have seen some diabetes patients dying because of the adverse side effects of the drugs for controlling diabetes and hypertension. Blood plasma can retain only 2% of sugar. When the sugar concentration rises beyond this 2%; blood becomes thick and it exerts pressure at the wall of the blood vessels. The proteins of the blood come out of the blood to provide space for the extra sugar and thereby pH of the blood relatively falls. This condition induces blood pressure many a times. Treating diabetes patients with allopathic drugs is not a desirable thing (Brady Pa., Terzic A (1998) The sulfonylurea controversy: more questions from heart. J Am Coll Cardiol 31(5):950-6). The side effects of allopathic medicines can be seen widely in diabetes patients. Diabetes patients are left with no option: if they do not control their blood sugar they may die of some or other physiological problems and if they take medicines their blood sugar is temporarily controlled but actually they are becoming severe diabetic from mild, as their doses of the medicine always increase. Medicines are to decrease the disease not to increase it. Oral hypoglycemic drugs (OHG) accelerate CAD (coronary artery disease). Inappropriate dosage of drugs results in unstable cellular glucose metabolism and this causes intracellular hypoxia. Besides these harmful effects, OHG are expensive also. This invention provides a healthy alternative to the diabetes treatment. Nowhere in the world, soybean alone is used as an effective blood sugar regulator. By developing the embryo of the soybean seeds, many physiological changes occur in the seeds. Soaking is the method applied here to achieve the embryo development of the soybean seeds. The basal levels of many blood sugar regulating compounds increase when embryo is developed. New molecules synthesize during embryo development (commonly known as germination). I hypothesize that some of the synthesized new molecules regulate the blood sugar by regulating insulin's optimum physiologic actions. One of such new molecule is nodule phosphatidylinositol 3 kinase. My hypothesis is based on the research work already done on the importance of PI3K in diabetes. Cloning and characterization of soybean PI3K was reported by Hong Z. and Verma D.P in 1994. It was also reported by them that this enzyme has not been characterized in any other plant and genes encoding the nodule form of PI3K is highly expressed in young root nodules. The polypeptides encoded by soybean PI3K cDNAs (both PI3Ks, root PI3K and nodule PI3K) show significant sequence homology (5040% similarity and 2040% identity) to mammalian and yeast PI3K (HongZ, Verma DP (1994) A phosphatidylinositol 3-kinase is induced during soybean nodule organogenesis and is associated with membrane proliferation. Proc NatI Acad Sci USA 91(20): 9617-21). Baumgartener J.W. in 2003 reported that PI3K is central to mediating insulin's action. He reported that inhibition of PI3K activity results in a blockade of insulin signaling including glucose uptake and glycogen synthesis (Baumgartener JW (2003) SHIP2: an emerging target for the treatment of type 2 diabetes mellitus. Curr Drug Targets Immune Endocr Metabol Disord 3(4): 291-8 ). Hence on the basis of these and other scientific reports (references in appendix), I feel that PI3K present in soybean is playing an important role in the treatment of diabetes. What is producing high blood sugar regulating property during embryo development is a big question for me. Presently on the basis of my experimental observations and available scientific reports, I feel that enrichment of many phytochemicals during germination makes soybean seeds an effective blood sugar regulator.

### BRIEF SUMMARY OF THE INVENTION

This invention relates to a blood sugar regulating product from soybean seeds which is equally or more effective than OHG. When embryo of the soybean seeds is developed; a high blood sugar regulating property is produced. This product will control the blood sugar as good as the OHG, and it will be free from the adverse side effects of OHG on health. Rather it helps in overcoming other diabetes related health problems like hypertension, heart diseases, high cholesterol, fatigue, joint pains, weak visions, weak memory and so on. Besides its efficacy, it is a wholesome natural food without addition of any synthetic chemical. The big problem I faced is what name shall I give to this natural product? A drug ? Or A food Supplement? Whatever name given to this product the truth is that it regulates blood sugar in many diabetes patients effectively when taken alone as a medicine. When taken with medicine its effect is not so obvious. The object of the invention is to provide a safe, cost effective and an effective blood sugar regulator.

### BRIEF DESCRIPTION OF THE SEVERAL VIEWS OF THE DRAWING

“Not applicable”.

### DETAILED DESCRIPTION OF THE INVENTION

During my post doctoral research work (1993-1998) on genetic aspects of type-I diabetes, I was able to interact with diabetes type H patients also. I came to know of the adverse side effects on health of the existing hypoglycemic drugs and the fact that most of them were suffering from the problem of mild to severe acidity. I thought insulin might not be the only problem and there could be some physiological disorder resulting into insulin's malfunctioning causing high blood sugar. I observed that by taking oral hypoglycemic drugs (OHG) and insulin regularly the patients were changing from mild to severe to chronic. I also observed that when OHG are initiated as a medicine for controlling blood sugar, usually the dose of such medicines increase with the passage of time; where as the required dose of the medicines should ideally decrease with the passage of time. The increase in the dose clearly indicates that disease is worsening. My aim was to develop an alternate therapy with equal or high efficacy and with no adverse health effects. In order to ensure this I looked for naturally existing eatables. After the completion of my formal postdoctoral research in 1998, I made a thorough study of human physiology, particularly metabolism. On the basis of my understanding of the possible reasons of diabetes I looked for certain ingredients in various plant products (calcium, phosphate, vitamins of B group and essential amino acids particularly leucine and lycine). By making use of the existing literature about the availability of ingredients in the plant products, I made a list of probable natural antidiabetes agents. This consisted mainly of alkaline foods. I was able to prepare a package of natural eatables effective in controlling blood sugar in diabetes patients. It was an advantage to me that some diabetes patients voluntarily accepted my advice, relied completely on my package and needed no other hypoglycemic drug. But still I was not satisfied, for I wanted a single medicine equal or more effective than hypoglycemic drugs and without any harmful health effect. I wanted to achieve such a product, which could act as a substitute to drugs with equal or more efficacy. In course of my experiments with various plant products, I investigated soybean's efficacy also as it contains most of the desirable ingredients including all eight essential amino acids. On soybean I conducted my first experiment with soybean milk on a diabetes patient and the result appeared to be unbelievably good to me. Patients took two doses (each dose of 250 ml) of soybean milk one in the morning before food and one in the evening before dinner as a medicine. Soybean seeds were soaked for 8 to 12 hours, soaked seeds were ground in fine paste, a milk was prepared from this paste by adding water into it, finally this milk was heated up till boiling. This boiled milk after getting cooled was filtered. This filtered milk was the final form of medicine for patients. Later I carried investigations on 17 volunteer type II diabetes patients and got very encouraging results (table-1& table-1a).

These were enough for me to believe in three things: Soybean milk is an effective anti-diabetes agent. Soybean milk is a blood sugar regulator. It always brings the blood sugar level towards normal blood sugar level of an individual. If the blood sugar is greater than the normal then it causes decrease in it and if the blood sugar is less than the normal then it causes increase in it. However the number of data for increasing the blood sugar level of hypoglycemic patients is not enough. Greater the blood sugar level greater is the fall due to soybean milk.

Then I filed a patent for this invention (904/DEL/2002). In order to find the value addition step of my process, I checked whether raw soybean powder shows any effective anti-diabetes property or not. I found that unsoaked soybean seeds (in powder form) brought almost no significant fall in high blood sugar levels. I realized that it is the embryo development of the seed, which is responsible for producing an amazing blood sugar regulating property in soybean seeds. Hence I soaked the soybean seeds, left it for little bit of germination and later dried it in the sun. Then I ground this dried powder which was my medicine for giving to my volunteer diabetes patients (table-2&2a). I observed high anti-diabetes property of this powder. This experimental finding confirmed my understanding that soaking and germination is producing high blood sugar regulating property in soybean seeds. Then I filed, in May 2003 another patent for this dry powder form of soaked and germinated soybean seeds in India. Later the effect of soaked and germinated soybean dry powder on 18 volunteer diabetes patients was investigated for three months. I observed and patients experienced that this powder is more effective than oral hypoglycemic drugs. In total I found four of the above diabetes patients were totally revived. But yet I do not have sufficient data to claim that it can cure the diabetes completely after intake of this powder or solution prepared from soaked soybean seeds for some duration. But these four examples enable me to claim that this medicine not only regulates blood sugar but it can also cure some of the diabetes patients.

This was not the end of my research. I was excited to know what is happening during germination, which is converting simple soybean seeds into highly effective anti-diabetes medicine. I put all my theories on search. Since then I got research papers on the basis of which I can give theoretical explanation for the development of anti-diabetic property in soybean seeds. By studying these papers I can construct the theory which can give the possible explanation for the development of anti-diabetes property in soybean seeds during soaking and germination. I can think of three possibilities, which could be responsible for the development of the anti-diabetes property in soybean seeds during soaking and germination: that synthesis of Phosphatidyllnositol 3 Kinase (PI3K) in soybean seed during germination is central to mediating insulin's metabolic effect. PI3K catalyzes the generation of phosphatidylinositol (3,4,5) triphosphate (PIP(3)). Inhibition of PI3K activity results in blockade of insulin signaling including glucose uptake and glycogen synthesis. This, PIP (3) is a critical mediator of insulin action. Either this soybean PI3K is catalyzing the reactions to facilitate the proper reactions to achieve insulin's physiologic expression or it is helping in synthesis of appropriate phosphatidylinositol derivative which might be mediating insulin's physiologic expression. I am evaluating both the possibilities (ref. 1-5). That synthesis of [-chiro inositol in soybean seeds during embryo development could be producing antidiabetes property in soaked and germinated soybean seeds. (ref.-6,7) Enhancement of vitamins during soaking and germination of soybean seed, particularly vitamins of B group (niacin and riboflavin) could be playing important roles. (a) The vitamins enriched soaked and germinated soybean seeds could be acting as a good superoxide scavengers. Recent studies report that overproduction of superoxide by the mitochondrial electron transport chain seems to be the first and keyevent in the activation of all other pathways involved in the pathogenesis of diabetic complications (ref-8). (b) intracellular concentration of NAD (nicotinamide adenine dinucleotide) is depleted in diabetes (ref-8 ) which slows the rate of glycolysis. Niacin, increased during embryo development of soybean seeds, could be increasing the intracellular level of NAD and hence increasing the rate of glycolysis. Vitamins are organic nutrients that are required in small quantities for a variety of biochemical functions, and which generally, cannot be synthesized by the body and must therefore be supplied by the diet.

It was observed that soaking and germination produce high blood sugar regulating property in simple soybean (Glycine max) seeds. This natural therapy overcomes the harmful side effects on health associated with OHG besides being cost effective. This will change the scenario of the treatment of type-2 diabetes patients. Earlier soybean was known for high fibre content and isoflavones as active ingredients for diabetes. This invention will give a new direction to the treatment of diabetes.

References

1. Hong Z, Verma D P (1994) A phosphatidylinositol 3-kinase is induced during soybean nodule organogenesis and is associated with membrane proliferation. Proc Natl] Acad Sci USA 91(20): 9617-21
2. Lochhead P A, Coghlan M, Rice S Q, Sutherland C (2001) Inhibition of GSK-3 selectively reduces glucose6 -phosphatase and phosphatase and phosphoenolypyruvate carboxykinase gene expression. Diabetes 50(5): 93746
3. Baumgartener J W (2003) SHIP2: an emerging target for the treatment of type 2 diabetes mellitus. Curr Drug Targets Immune Endocr Metabol Disord 3(4): 291-8
4. Bouzakri K, Roques M, Gual P, Espinosa S, Guebre-Egziabher F, Riou J P, Laville M, Le Marchand-Brustel Y, Tanti J F, Vidal H (2003) Reduced activation of phosphatidylinositol-3 kinase and increased serine 636 phosphorylation of insulin receptor substrate-1 in primary culture of skeletal muscle cells from patients with type 2 diabetes. Diabetes 52(6): 1319-25
5. Hori H, Sasaoka T, Ishihara H, Wada T, Murakami S, Ishiki M, Kobayashi M (2002) Association of SH2-containing inositol phosphatase 2 with the insulin resistance of diabetic db/db mice. Diabetes 51(8): 2387-94
6. Lamer J (2002) D-chiro-inositol-its functional role in insulin action and Its deficit in insulin resistance. Int J Exp Diabetes Res 3(1): 47-60
7. Guoqiao Jiang, Ammulu Hari Krishnan, Yong-Woong Kim, Thomas J. Wacek, and Hari B. Krishnan (2001) Functional myo-Inositol Dehydrogenase Gene Is Required for Efficient Nitrogen Fixation. Journal of Bacteriology 183 (8): 2595-2604

8. Ceriello A (2003) New insights on oxidative stress and diabetic complications may lead to a “causal” antioxidant therapy. Diabetes Care 26(5): 1589-96

TABLE 1
Effect of 250 ml Soaked Soybean solution (SSS) on the
blood sugar after two hours of its intake
(on group-1 patients)

| | | | | Blood | |
|---|---|---|---|---|---|
| | | | Random Blood | Sugar in | Reduction |
| | Age at | | sugar (after six | mg/dl after | in blood |
| | detection | Pres- | hours of the lunch) | two hours | sugar in |
| pa- | of | ent | in mg/dl when they | of intake | mg/dl due |
| tient | diabetes | Age | were on OHG | of SSS | to SSS |
| 1 | 45 | 50 | 134 | 123 | 11 |
| 2 | 63 | 70 | 294 | 109 | 185 |
| 3 | 36 | 58 | 141 | 110 | 31 |
| 4 | 68 | 70 | 477 | 409 | 68 |
| 5 | 46 | 46 | 355 | 290 | 65 |
| 6 | 45 | 52 | 249 | 172 | 77 |
| 7 | 43 | 48 | 170 | 140 | 30 |
| 8 | 37 | 40 | 196 | 106 | 90 |
| 9 | 38 | 44 | 121 | 104 | 17 |
| 10 | 36 | 45 | 166 | 123 | 43 |
| 11 | 40 | 45 | 96 | 75 | 21 |
| 12 | 43 | 43 | 152 | 115 | 37 |
| 13 | 40 | 49 | 232 | 214 | 18 |
| 14 | 38 | 42 | 135 | 84 | 51 |
| 15 | 40 | 43 | 89 | 63 | 26 |
| 16 | 43 | 45 | 86 | 116 | −30 |
| 17 | 30 | 37 | 76 | 84 | −8 |

OHG = Oral Hypoglycemic Drug
Random Blood Sugar = Blood sugar taken after six hours of food.

TABLE 1 (a)
The effect of Soaked Soybean Solution (SSS) on blood sugar for four months on
patients taking SSS twice a day (250 ml per dose) after stopping OHG.
(on group-1 patients)

| | Previous random blood Sugar | Blood Sugar in mg/dl after the start of SSS as a medicine and | | | |
|---|---|---|---|---|---|
| | in mg/dl when the patient was | after stopping other OHG for controlling blood sugar | | | |
| | taking OHG medicine for | August 2002 | | | |
| | controlling blood sugar | Fasting | | | |
| Patients of | Detected Diabetic for the first | After 10 | Sept 2002 | October 2002 | November 2002 |
| Table 1 | time in August 2002. | Days | Fasting | Fasting | Fasting |
| 5 | 355 | 121 | 103 | 109 | 106 |
| 13 | 232 | 134 | 100 | 117 | 126 |
| 11 | 96 | 89 | 108 | 118 | 106 |
| 12 | 152 | 118 | 115 | 92 | 108 |

OHG = Oral Hypoglycemic Drug
Random Blood Sugar = Blood sugar taken after six hours of food

TABLE 2
Effect of Soaked Soybean Powder (SSP) on blood sugar after two hours of its intake
after mixing it in a half glass of lukewarm water
(on group-2 patients)

| | | | | Random Blood | | | |
|---|---|---|---|---|---|---|---|
| | | | | sugar** (after six | | Blood Sugar in | Reduction in |
| | | Age at | | hours of lunch) in | | mg/dl after two | blood sugar |
| | | detection of | Present | mg/dl when they were | Dosage of SSP | hours of intake of | in mg/dl due |
| Patient | Sex | diabetes | Age | on OHG* | in gms | SSP | to SSP |
| 1 | M | 40 | 46 | 264 | 15 | 165 | 99 |
| 2 | M | 44 | 47 | 315 | 15 | 197 | 118 |
| 3 | F | 44 | 45 | 145 | 7.5 | 128 | 17 |
| 4 | M | 45 | 52 | 230 | 15 | 155 | 75 |
| 5 | M | 42 | 42 | 168 | 15 | 98 | 70 |
| 6 | M | 57 | 59 | 235 | 15 | 82 | 153 |
| 7 | M | 45 | 47 | 149 | 15 | 109 | 40 |
| 8 | M | 42 | 47 | 294 | 7.5 | 159 | 135 |
| 9 | M | 37 | 51 | 173 | 15 | 106 | 67 |
| 10 | M | 52 | 58 | 237 | 15 | 156 | 81 |
| 11 | F | 37 | 51 | 321 | 15 | 149 | 172 |
| 12 | F | 56 | 58 | 212 | 15 | 147 | 65 |
| 13 | M | 44 | 46 | 316 | 15 | 305 | 11 |
| 14 | M | 51 | 59 | 253 | 15 | 173 | 80 |
| 15 | F | 46 | 51 | 384 | 15 | 294 | 90 |
| 16 | M | 40 | 45 | 191 | 15 | 120 | 71 |
| 17 | F | 43 | 47 | 145 | 7.5 | 128 | 17 |
| 18 | F | 48 | 50 | 262 | 15 | 167 | 95 |

OHG = Oral Hypoglycemic Drug
Random Blood Sugar = Blood sugar taken after six hours of food

TABLE 2 (a)
The effect of Soaked Soybean Powder (SSP) on blood sugar for three months on
patients taking SSP twice a day (15 gms per dose) after stopping OHG.
(on group-2 patients)

| | | | | Previous blood | | Blood sugar in mg/dl after patients started SSP only | | | | | | | |
|---|---|---|---|---|---|---|---|---|---|---|---|---|---|
| | | | | sugar in mg/dl when | | as a blood sugar controlling medicine | | | | | | | |
| Patients | | | | patients were taking | | After 15 | | After 1 | | After 2 | | After 3 | |
| of | Age | | | OHG drugs | | Days | | Month | | months | | months | |
| Table-2 | ADD | ATM | PA | F | PP | F | PP | F | PP | F | PP | F | PP |
| 1 | 40 | 40 | 46 | 140 | 264 | 130 | 243 | 112 | 153 | 95 | 210 | 96 | 140 |
| 2 | 44 | 44 | 47 | 148 | 361 | 135 | 254 | 140 | 232 | 130 | 220 | 122 | 212 |
| 3 | 44 | 44 | 45 | 150 | 210 | 118 | 145 | 108 | 122 | 85 | 130 | 80 | 120 |
| 4 | 45 | 45 | 52 | 190 | 235 | 150 | 191 | 134 | 150 | 121 | 146 | 116 | 144 |
| 5 | 42 | 42 | 42 | 120 | 168 | 110 | 168 | 97 | 151 | 88 | 134 | 73 | 106 |
| 6 | 57 | 57 | 59 | 188 | 210 | 96 | 150 | 92 | 143 | 84 | 132 | 85 | 124 |
| 7 | 45 | 45 | 47 | 119 | 149 | 110 | 132 | 87 | 122 | 93 | 144 | 102 | 108 |
| 8 | 42 | 42 | 47 | 198 | 294 | 179 | 271 | 164 | 258 | 139 | 237 | 152 | 240 |
| 9 | 37 | 37 | 51 | 106 | 173 | 96 | 173 | 87 | 118 | 106 | 127 | 84 | 120 |
| 10 | 52 | 52 | 58 | 155 | 237 | 156 | 237 | 157 | 189 | 158 | 190 | 131 | 189 |
| 11 | 37 | 37 | 51 | 152 | 321 | 148 | 165 | 140 | 188 | 139 | 155 | 126 | 150 |
| 12 | 56 | 56 | 58 | 174 | 325 | 194 | 310 | 168 | 201 | 175 | 295 | 164 | 210 |
| 13 | 44 | 44 | 46 | 277 | 316 | 262 | 298 | 251 | 284 | 236 | 273 | 186 | 239 |
| 14 | 51 | 51 | 59 | 168 | 293 | 174 | 281 | 158 | 264 | 163 | 273 | 157 | 261 |
| 15 | 46 | 46 | 51 | 221 | 384 | 190 | 35 | 185 | 321 | 156 | 271 | — | — |
| 16 | 40 | 40 | 45 | 150 | 191 | 161 | 210 | 134 | 150 | 121 | 146 | 124 | 160 |
| 17 | 43 | 44 | 47 | 130 | 173 | 107 | 112 | 108 | 122 | 85 | 130 | 80 | 120 |
| 18 | 48 | 48 | 50 | 156 | 262 | 145 | 232 | 133 | 195 | 121 | 155 | 107 | 164 |

ADD = Age at Diabetes Detected
ATM = Age at Medicine (OHG) Taken for control of the blood sugar
PA = Present Age

dinucleotide ) is depleted in diabetes ( ref-8 ) which slows the rate of glycolysis. Niacin, increased during embryo development of soybean seeds, could be increasing the intracellular level of NAD and hence increasing the rate of glycolysis. Vitamins are organic nutrients that are required in small quantities for a variety of biochemical functions, and which generally, cannot be synthesized by the body and must therefore be supplied by the diet.

Apart from the above possible factors on the basis of published research papers, I also think on my own that during diabetes human body goes to lower energy state and when a diabetes patient takes soaked and germinated soybean powder the energy state of the body goes up. I think this low energy state of the human body is the vital factor for many physiological complications including reduced activation of many enzymes in the body.

### Table-1

Effect of 250 ml Soaked Soybean solution (SSS) on the blood sugar after two hours of its intake

### (on group- patients)

TABLE 1
Effect of 250 ml Soaked Soybean solution (SSS) on the blood sugar after two
hours of its intake (on group-1 patients)

| | | | Random Blood | | |
|---|---|---|---|---|---|
| | Age at | | sugar** (after six | Blood Sugar in | |
| | detection | | hours of the lunch) | mg/dl after two | Reduction in |
| | of | Present | in mg/dl when they | hours of intake | blood sugar in |
| patient | diabetes | Age | were on OHG* | of SSS | mg/dl due to SSS |
| 1 | 45 | 50 | 134 | 123 | 11 |
| 2 | 63 | 70 | 294 | 109 | 185 |
| 3 | 36 | 58 | 141 | 110 | 31 |
| 4 | 68 | 70 | 477 | 409 | 68 |
| 5 | 46 | 46 | 355 | 290 | 65 |
| 6 | 45 | 52 | 249 | 172 | 77 |
| 7 | 43 | 48 | 170 | 140 | 30 |
| 8 | 37 | 40 | 196 | 106 | 90 |
| 9 | 38 | 44 | 121 | 104 | 17 |
| 10 | 36 | 45 | 166 | 123 | 43 |
| 11 | 40 | 45 | 96 | 75 | 21 |
| 12 | 43 | 43 | 152 | 115 | 37 |
| 13 | 40 | 49 | 232 | 214 | 18 |
| 14 | 38 | 42 | 135 | 84 | 51 |
| 15 | 40 | 43 | 89 | 63 | 26 |
| 16 | 43 | 45 | 86 | 116 | −30 |
| 17 | 30 | 37 | 76 | 84 | −8 |

OHG* = Oral Hypoglycemic Drug
Random Blood Sugar** = Blood sugar taken after six hours of food.

1. A blood sugar regulating product obtainable from soybean seeds by a process comprising one or more than one of the following steps
a) soaking the soybean seeds to achieve the embryo development,
b) drying the soaked soybean seeds to reduce the water content of seeds,
a) grinding the seeds to get the desired product.

2. A product obtainable by a process as claimed in claim 1 by any alteration in the process made e.g. achieving embryo development by any means, mixing of any other ingredient for the change of the taste or flavor or for any other reason so that blood sugar regulating property is not lost.

3. A blood sugar regulating product obtainable by a process that involves isolation, extraction or enrichment of phosphatidylinositol 3 kinase or its corresponding gene in part or full from soybean.

4. A product obtainable from soaked and/or germinated soybean seeds by a process involving isolation, extraction or enrichment of any ingredient (e.g. Inositols such as phosphatidylinositol 3-phosphate, myoinositols, lipid polysaccharide, D-chiro inositol, dehydrogenases such as tyrosine dehydrogenase, myoinositol dehydrogenase in part or full, with the purpose of achieving blood sugar regulating property.
